(19) United States
(12) Patent Application Publication
(10) Pub. No.: US 2010/0076003 A1
(43) Pub. Date: Mar. 25, 2010

(54) 5-OXAZOLIDIN-2-ONE SUBSTITUTED 1,3,8-TRIAZASPIRO[4.5]DECAN-4-ONE DERIVATIVES USEFUL AS ORL-1 RECEPTOR MODULATORS

(76) Inventors: Kathleen BATTISTA, Williamstown, NJ (US); Gilles Bignan, Bridgewater, NJ (US); Peter J. Connolly, New Providence, NJ (US); Tina Morgan Ross, Royerford, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/479,103

(22) Filed: Jun. 5, 2009

Related U.S. Application Data
(60) Provisional application No. 61/098,364, filed on Sep. 19, 2008.

(51) Int. Cl. A61K 31/438; C07D 471/10

(52) U.S. Cl. 514/278; 546/20

(57) ABSTRACT

The present invention is directed to novel 5-oxazolidin-2-one substituted 1,3,8-triazaspiro[4.5]decan-4-one derivatives useful in the treatment of disorders and conditions mediated by the ORL-1 receptor. The present invention is further directed to processes for the preparation of said derivatives, pharmaceutical compositions comprising said derivatives and methods for the treatment of ORL-1 mediated disorders and conditions.

### CROSS-REFERENCE TO RELATED APPLICATIONS

The present application claims the benefits of the filing of U.S. Provisional Application No. 61/098,364 filed Sep. 19, 2008. The complete disclosures of the aforementioned related patent applications are hereby incorporated herein by reference for all purposes.

### FIELD OF THE INVENTION

The present invention is directed to novel 5-oxazolidin-2-one substituted 1,3,8-triazaspiro[4.5]decan-4-one derivatives useful in the treatment of disorders and conditions mediated by the ORL-1 receptor. The present invention is further directed to processes for the preparation of said derivatives, pharmaceutical compositions comprising said derivatives and methods for the treatment of ORL-1 mediated disorders and conditions.

### BACKGROUND OF THE INVENTION

The ORL-1 (orphan opioid receptor) G-protein coupled receptor, also known as the nociceptin receptor (OP-4), was first reported in 1994, and was discovered based on its homology with the classic delta—(OP-1), mu—(OP-3), and kappa—(OP-2) opioid receptors. The ORL-1 G-protein coupled receptor does not bind opioid ligands with high affinity. The amino acid sequence of ORL-1 is 47% identical to the opioid receptors overall, and 64% identical in the transmembrane domains (Nature, 1995, 377, 532).

The endogenous ligand of ORL-1, known as nociceptin, a highly basic 17 amino acid peptide, was isolated from tissue extracts in 1995. It was named both nociceptin, because it increased sensitivity to pain when injected into mouse brain, and orphanin FQ (OFQ) because of the terminal phenylalanine (F) and glutamine (Q) residues that flank the peptide on the N— and C-termini respectively (PCT Publication, WO97/07212).

Nociceptin binding to ORL-1 receptors causes inhibition of cAMP synthesis, inhibition of voltage-gated calcium channels, and activation of potassium conductance. In vivo, nociceptin produces a variety of pharmacological effects that at times oppose those of the opioids, including hyperalgesia and inhibition of morphine-induced analgesia. Mutant mice lacking nociceptin receptors show better performance in learning and memory tasks. These mutant mice also have normal responses to painful stimuli.

The ORL-1 receptor is widely distributed/expressed throughout the human body, including in the brain and spinal cord. In the spinal cord, the ORL-1 receptor exists in both the dorsal and ventral horns, and precursor mRNA has been found in the superficial lamina of the dorsal horn, where primary afferent fibers of nociceptors terminate. Therefore, the ORL-1 has an important role in nociceptin transmission in the spinal cord. This was confirmed in recent studies wherein nociceptin, when given to mice by i.c.v. injection, induced hyperalgesia and decreased locomotor activity (Brit. J. Pharmacol. 2000, 129, 1261).

U.S. Pat. No. 6,043,366 issued Mar. 28, 2000 discloses 1,3,8,-triazaspiro(4,5)decan-4-one derivatives useful in treating disorders involving the Orphanin FQ receptor, and processes for their preparation.

There remains a need for small molecule modulators of the ORL-1 receptor, useful for the treatment of disorders and conditions mediated by the ORL-1 receptor, such as anxiety, depression, panic, dementia, mania, bipolar disorder, substance abuse, neuropathic pain, acute pain, chronic pain, migraine, asthma, cough, psychosis, schizophrenia, epilepsy, hypertension, obesity, eating disorders, cravings, diabetes, cardiac arrhythmia, irritable bowel syndrome, Crohn's disease, urinary incontinence, adrenal disorders, attention deficit disorder (ADD), attention deficit hyperactivity disorders (ADHD), Alzheimer's disease, for improved cognition or memory and for mood stabilization (Bignan G C, Connolly P J, Middleton S A, Recent advances towards the discovery of ORL-1 receptor agonists and antagonists, Expert Opinion on Therapeutic Patents, 2005, 15(4), 357-388).

PRD 3011 US and PCT

### SUMMARY OF THE INVENTION

The present invention is directed to compounds of formula (I)

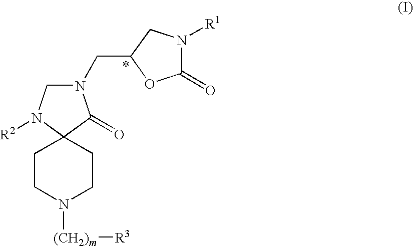

wherein

R¹ is selected from the group consisting of H, C₁₋₄alkyl, —C₁₋₄alkyl-OH and —C₁₋₄alkyl-O—C₁₋₄alkyl;

R² is selected from the group consisting of aryl, wherein the aryl is optionally substituted with one or more substituents independently selected from the group consisting of halogen, hydroxyl, C₁₋₄alkyl and C₁₋₄alkoxy;

m is an integer from 0 to 1;

R³ is selected from the group consisting of cycloalkyl, partially unsaturated carbocyclyl, aryl and heteroaryl; wherein the aryl or heteroaryl is optionally substituted with one or more (preferably one to two, more preferably one) substituents independently selected from the group consisting of halogen, hydroxyl, C₁₋₄alkyl and C₁₋₄alkoxy;

and enantiomers, diastereoisomers, esters, prodrugs, solvates and pharmaceutically acceptable salts thereof.

The present invention is further directed to processes for the preparation of the compounds of formula (I), as outlined in more detail herein. The present invention is further directed to a product prepared according to any of the processes described herein.

Illustrative of the invention is a pharmaceutical composition comprising a pharmaceutically acceptable carrier and a compound of formula (I) as described herein. An illustration of the invention is a pharmaceutical composition made by mixing a compound of formula (I) as described herein and a pharmaceutically acceptable carrier. Illustrating the invention is a process for making a pharmaceutical composition comprising mixing a compound of formula (I) as described herein and a pharmaceutically acceptable carrier.

Exemplifying the invention are methods of treating, preventing and/or ameliorating disorders and conditions mediated by the ORL-1 receptor comprising administering to a subject in need thereof a therapeutically effective amount of any of the compounds or pharmaceutical compositions described above.

An example of the invention is a method of treating a condition selected from the group consisting of anxiety, depression, panic, mania, dementia, bipolar disorder, substance abuse, neuropathic pain, acute pain, chronic pain, migraine, asthma, cough, psychosis, schizophrenia, epilepsy, hypertension, obesity, eating disorders, cravings, diabetes, cardiac arrhythmia, irritable bowel syndrome, Crohn's disease, urinary incontinence, adrenal disorders, attention deficit disorder (ADD), attention deficit hyperactivity disorder (ADHD), Alzheimer's disease, for improved cognition or memory and for mood stabilization, comprising administering to a subject in need thereof a therapeutically effective amount of any of the compounds or pharmaceutical compositions described above.

Another example of the invention is the use of any of the compounds described herein in the preparation of a medicament for treating: (a) anxiety, (b) depression, (c) panic, (d) mania, (e) dementia, (f) bipolar disorder, (g) substance abuse (h) neuropathic pain, (i) acute pain, (j) chronic pain, (k) migraine, (l) asthma, (m) cough, (n) psychosis, (o) schizophrenia, (p) epilepsy, (q) hypertension, (r) obesity, (s) eating disorders, (t) cravings, (u) diabetes, (v) cardiac arrhythmia, (w) irritable bowel syndrome, (x) Crohn's disease, (uy) urinary incontinence, (z) adrenal disorders, (aa) attention deficit disorder (ADD), (bb) attention deficit hyperactivity disorder (ADHD), (cc) Alzheimer's disease, for (dd)improved cognition, (ee) improved memory and (ff) mood stabilization, in a subject in need thereof.

### DETAILED DESCRIPTION OF THE INVENTION

The present invention is directed to novel 5-oxazolidin-2-one substituted 1,3,8-triazaspiro[4.5]decan-4-one derivatives useful for the treatment of disorders and conditions mediated by the ORL-1 receptor. More particularly, the present invention is directed to compounds of I formula (I)

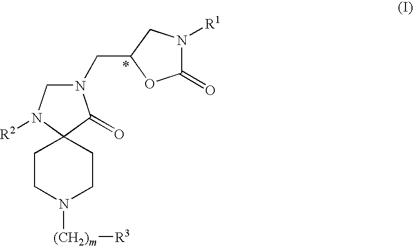

wherein R¹, R², R³ and are as herein defined; and enantiomers, diastereoisomers, esters, prodrugs, solvates and pharmaceutically acceptable salts thereof. The compounds of formula (I) are useful in the treatment of disorders mediated by the ORL-1 receptor. More particularly, the compounds of formula (I) are useful in the treatment of anxiety, depression, panic, mania, dementia, bipolar disorder, substance abuse, neuropathic pain, acute pain, chronic pain migraine, asthma, cough, psychosis, schizophrenia, epilepsy, hypertension, obesity, eating disorders, cravings, diabetes, cardiac arrhythmia, irritable bowel syndrome, Crohn's disease, urinary incontinence, adrenal disorders, attention deficit disorder (ADD), attention deficit hyperactivity disorder (ADHD), Alzheimer's disease, for improved cognition or memory and for mood stabilization. Preferably, the compounds of formula (I) are useful in the treatment of anxiety, depression, substance abuse, neuropathic pain, acute pain, chronic pain, migraine, cough, hypertension, cardiac arrhythmia, irritable bowel syndrome and Crohn's disease. More preferably, the compounds of formula (I) are useful the treatment of anxiety, depression, neuropathic pain, acute pain, chronic pain and migraine.

In an embodiment, the present invention is directed to compounds of formula (I) wherein R¹ is selected from the group consisting of hydrogen, C₁₋₂alkyl, C₁₋₂alkyl-OH and —CH₂CH₂OCH₃. In another embodiment of the present invention, R¹ is selected from the group consisting of hydrogen, C₁₋₂alkyl and —CH₂CH₂OCH₃. In another embodiment of the present invention, R¹ is selected from the group consisting of hydrogen, methyl and —CH₂CH₂OCH₃. In another embodiment of the present invention, R¹ is hydrogen.

In an embodiment of the present invention, R² is substituted phenyl. In another embodiment of the present invention, R² is phenyl; wherein the phenyl is optionally substituted with one to three (preferably one to two) halogen (preferably chloro or fluoro). In another embodiment of the present invention, R² is 4-fluorophenyl.

In an embodiment of the present invention, R³ is selected from the group consisting of cycloalkyl, partially unsaturated carbocyclyl and aryl; wherein the aryl is optionally substituted with C₁₋₂alkyl. In another embodiment of the present invention, R³ is selected from the group consisting of cyclooctyl, acenaphthenyl and aryl; wherein the aryl is optionally substituted with methyl. In another embodiment of the present invention, R³ is selected from the group of cyclooctyl, naphthyl, and 8-methyl-naphthy-1-yl. In another embodiment of the present invention, R³ is cyclooctyl. In another embodiment of the present invention, R³ is 8-methyl-naphthy-1-yl. In another embodiment of the present invention, R³ is acenaphthenyl.

In an embodiment of the present invention, m is 1. In another embodiment of the present invention, m is 0.

In another embodiment, the present invention is directed to any single compound or subset of compounds selected from the group consisting of

8-(R)-Acenaphthen-1-yl-1-(4-fluoro-phenyl)-3-(S)-(2-oxo-oxazolidin-5-ylmethyl)-1,3,8-triaza-spiro[4.5]decan-4-one;

1-(4-Fluoro-phenyl)-8-(8-methyl-naphthalen-1-ylmethyl)-3-(R)-(2-oxo-oxazolidin-5-ylmethyl)-1,3,8-triaza-spiro[4.5]decan-4-one;

8-Cyclooctylmethyl-1-(4-fluoro-phenyl)-3-(2-oxo-oxazolidin-5-ylmethyl)-1,3,8-triaza-spiro[4.5]decan-4-one;

1-(4-Fluoro-phenyl)-8-(8-methyl-naphthalen-1-ylmethyl)-3-(S)-(3-methyl-2-oxo-oxazolidin-5-ylmethyl)-1,3,8-triaza-spiro[4.5]decan-4-one; and

1-(4-Fluoro-phenyl)-3-[3-(2-methoxy-ethyl)-2-oxo-oxazolidin-5-ylmethyl]-8-(8-methyl-naphthalen-1-ylmethyl)-1,3,8-triaza-spiro[4.5]decan-4-one;

and enantiomers, diastereoisomers, esters, prodrugs, solvates and pharmaceutically acceptable salts thereof.

In an embodiment of the present invention, when R³ is aryl, heteroaryl or cycloalkyl, then m is 1. In another embodiment, the present invention is directed to compounds of formula (Ia)

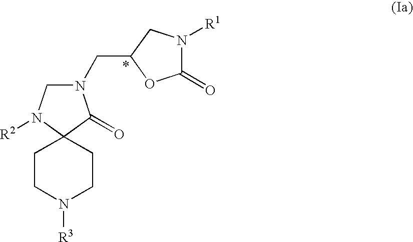

wherein R¹ is selected from the group consisting of hydrogen, C₁₋₂alkyl, C₁₋₂alkyl-OH and —CH₂CH₂OCH₃;

R² is phenyl; wherein the phenyl is optionally substituted with one to three halogen;

R³ is selected from the group consisting of partially unsaturated carbocyclyl, aryl and heteroaryl; wherein the aryl or heteroaryl is optionally substituted with C₁₋₂alkyl;

and enantiomers, diastereoisomers, esters, prodrugs, solvates and pharmaceutically acceptable salts thereof.

As used herein, the notation “*” shall denote the presence of a stereogenic center.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

In an embodiment of the present invention, the stereocenter as denoted by the “*” symbol in the oxazolidinone portion of the compounds of formula (I)

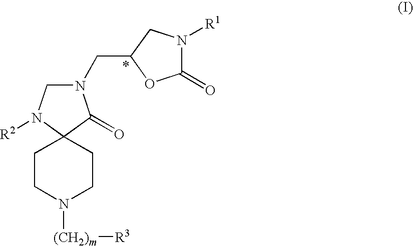

is present in the R stereo-configuration, as denoted below

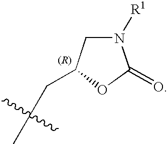

In another embodiment of the present invention, the stereocenter as denoted by the “*” symbol in the oxazolidinone portion of the compounds of formula (I) is present in the S stereoconfiguration, as denoted below:

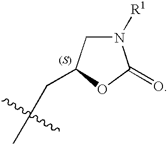

One skilled in the art will recognize that the R³ substituent may additionally contain more or none stereocenters. The present invention shall include all stereoconfigurations thereof.

Additional embodiments of the present invention, include those wherein the substituents selected for one or more of the variables defined herein (i.e. wherein m, R¹, R² and R³) are independently selected to be any individual substituent or any subset of substituents selected from the complete list as defined herein.

In another embodiment, the present invention is directed to any single compound or subset of compounds selected from the representative compounds of formula (I) listed in Table 1. Unless otherwise noted, wherein a stereocenter is present in the listed compound, the compound was prepared as a mixture of stereo-configurations. Where a stereocenter is present, and the compound was prepared in an excess of one of the stereo-isomers, the S*— and R* designations are intended to indicate that the exact stereo-configuration of the center has not been determined.

TABLE 1
Representative Compounds of Formula (I)

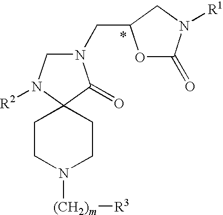

| | | | | | Stereo- |
|---|---|---|---|---|---|
| ID | R¹ | R² | R³ | m | center* |
| 1 | H | 4-fluoro-phenyl | (R)- | 0 | S |
| | | | acenaphthenyl | | |
| 2 | H | 4-fluoro-phenyl | 8-methyl- | 1 | R |
| | | | naphthyl | | |
| 3 | H | 4-fluoro-phenyl | cyclooctyl | 1 | R |
| 4 | methyl | 4-fluoro-phenyl | 8-methyl- | 1 | S |
| | | | naphthyl | | |
| 5 | 2-methoxy | 4-fluoro-phenyl | 8-methyl- | 1 | R |
| | ethyl | | naphthyl | | |

As used herein, unless otherwise noted, “halogen” shall mean chlorine, bromine, fluorine and iodine.

As used herein, unless otherwise noted, the term “alkyl”, whether used alone or as part of a substituent group, shall include straight chains. For example, alkyl radicals include methyl, ethyl, n-propyl, n-butyl, n-pentyl, n-hexyl, and the like.

As used herein, unless otherwise noted, “alkoxy” shall denote an oxygen ether radical of any of the above described straight chain alkyl groups. For example, methoxy, ethoxy, n-propoxy, n-butoxy, n-pentoxy, n-hexyloxy and the like.

As used herein, when used with terms such as alkyl, alkoxy, and the like, the prefix C_{X-Y} wherein X and Y are integers, shall denote an alkyl chain of between X and Y carbon atoms. For example, C₁₋₄alkyl, shall denote an alkyl chain of one to four carbon atoms.

As used herein, unless otherwise noted, “aryl” shall refer to carbocyclic aromatic groups. Suitable examples include but are not limited to phenyl, naphthyl, and the like.

As used herein, unless otherwise noted, the term “cycloalkyl” shall mean any stable 3-8 membered monocyclic, saturated ring system, for example cyclopropyl, cyclobutyl, cyclopentyl, cyclohexyl, cycloheptyl and cyclooctyl.

As used herein, unless otherwise noted, the term “carbocyclyl” shall mean any four to fourteen membered, preferably five to thirteen membered, more preferably five to ten membered monocyclic, bicyclic or tricyclic, carbon based ring structure. Similarly, unless otherwise noted, the term “partially unsaturated carbocyclyl” shall mean any five to fourteen, preferably five to thirteen, more preferably five to ten, membered monocyclic, bicyclic or tricyclic, carbon based ring structure containing at least one unsaturated (double or triple) bond. Suitable examples of partially unsaturated carbocyclyl groups include 1-acenaphthenyl,

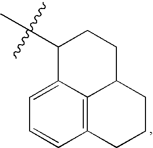

and the like.

As used herein, unless otherwise noted, “heteroaryl” shall denote any five or six membered monocyclic aromatic ring structure containing at least one heteroatom selected from the group consisting of O, N and S, optionally containing one to three additional heteroatoms independently selected from the group consisting of O, N and S; or a nine or ten membered bicyclic aromatic ring structure containing at least one heteroatom selected from the group consisting of O, N and S, optionally containing one to four additional heteroatoms independently selected from the group consisting of O, N and S. The heteroaryl group may be attached at any heteroatom or carbon atom of the ring such that the result is a stable structure.

Examples of suitable heteroaryl groups include, but are not limited to, pyrrolyl, furyl, thienyl, oxazolyl, imidazolyl, purazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzthiazolyl, purinyl, quinolizinyl, quinolinyl, isoquinolinyl, isothiazolyl, cinnolinyl, phthalazinyl, quinazolinyl, quinoxalinyl, naphthyridinyl, pteridinyl, and the like.

When a particular group is “substituted” (e.g., cycloalkyl, aryl, heteroaryl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

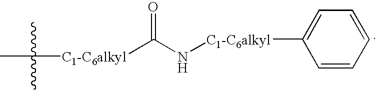

Abbreviations used in the specification, particularly the Schemes and Examples, are as follows:
BOC or Boc or t-Boc=tert-Butoxycarbonyl (i.e. —C(O)—O—C(CH₃)₃)
BOC-anhydride=Di-tert-butyl dicarbonate
CBz=Benzyloxycarbonyl (i.e —C(O)—O—CH₂—C₆H₅)
DCE=1,1-Dichloroethane
DIPEA or DIEA=Diisopropylethylamine
DMF=N,N-Dimethylformamide
DMSO=Dimethylsulfoxide
EGTA=Ethylene glycol-O,O′-bis(2-aminoethyl)-N,N,N′,N′-tetracacetic acid
Fmoc=9-Fluorenylmethoxycarbonyl
HPLC=High Pressure Liquid Chromatography
KO-t-Bu=Potassium t-butoxide
LiHMDS=Lithium bis(trimethylsilyl)amide
NaHMDS=Sodium bis(trimethylsilyl)amide
NMP=N-methyl-2-pyrrolidinone
TFA=Trifluoroacetic Acid
THF=Tetrahydrofuran
Triflate=Trifluoromethylsulfonyloxo (i.e. —O—SO₂—CF₃)
Tris HCl or Tris-Cl=Tris[hydroxymethyl]aminomethyl hydrochloride

As used herein, unless otherwise noted, the term “isolated form” shall mean that the compound is present in a form which is separate from any solid mixture with another compound(s), solvent system or biological environment. In an embodiment of the present invention, the compound of formula (I) is present in an isolated form.

As used herein, unless otherwise noted, the term “substantially pure compound” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment of the present invention, the compound of formula (I) is present as a substantially pure compound.

As used herein, unless otherwise noted, the term “substantially free of a corresponding salt form(s)” when used to described the compound of formula (I) shall mean that mole percent of the corresponding salt form(s) in the isolated compound of formula (I) is less than about 5 mole percent, preferably less than about 2 mole percent, more preferably, less than about 0.5 mole percent, most preferably less than about 0.1 mole percent. In an embodiment of the present invention, the compound of formula (I) is substantially free of corresponding salt forms.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that the amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As used herein, unless otherwise noted, the terms “treating”, “treatment” and the like, shall include the management and care of a subject or patient (preferably mammal, more preferably human) for the purpose of combating the disease, condition, or disorder and includes the administration of a compound of the present invention to prevent the onset of the symptoms or complications, alleviate the symptoms or complications, or eliminate the disease, condition, or disorder.

As used herein, unless otherwise noted, the term “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition. One skilled in the art will recognize that wherein the present invention are directed to methods of prevention, subjects in need of thereof (i.e. subjects in need of prevention) shall include any subject or patient (preferably a mammal, more preferably a human) who has experienced or exhibited at least one symptom of the disorder or condition to be prevented.

Further, in regards to prevention of a disorder or condition as herein described, a subject in need thereof may additionally be a subject (preferably a mammal, more preferably a human) who has not exhibited any symptoms of the disorder or condition, but who has been deemed by a physician, clinician or other medical profession to be at risk of developing said disorder or condition. For example, the subject may be deemed at risk of developing a disorder or condition (and therefore in need of prevention or preventive treatment) as a consequence of the subject's medical history, including, but not limited to, family history, pre-disposition, co-existing disorders or conditions, genetic testing, and the like.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems. One skilled in the art will further recognize that wherein two consecutive reaction or process steps are run without isolation of the intermediate product (i.e. the product of the first of the two consecutive reaction or process steps), then the first and second reaction or process steps may be run in the same solvent or solvent system; or alternatively may be run in different solvents or solvent systems following solvent exchange, which may be completed according to known methods.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any range therein.

Examples of suitable solvents, bases, reaction temperatures, and other reaction parameters and components are provided in the detailed descriptions which follows herein. One skilled in the art will recognize that the listing of said examples is not intended, and should not be construed, as limiting in any way the invention set forth in the claims which follow thereafter.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

As used herein, unless otherwise noted, the term “protecting group” shall mean a group which may be attached to a reactive terminal group (for example a nitrogen r oxygen atom) to protect said group from participating in a reaction, and which may be readily removed following the reaction. In an example, the protecting group may be a “nitrogen protecting group”, a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1999.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1999. The protecting groups may be removed at a convenient subsequent stage using methods known from the art.

Where the processes for the preparation of the compounds according to the invention give rise to a mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α−obs]/[α−max])×100.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotine acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

Compounds of formula (I) of the present invention may be prepared according to the processes as described in Schemes 1 through 5, as outlined in more detail below. More particularly, the compounds of formula (I) may be prepared as outlined in Scheme 1, below.

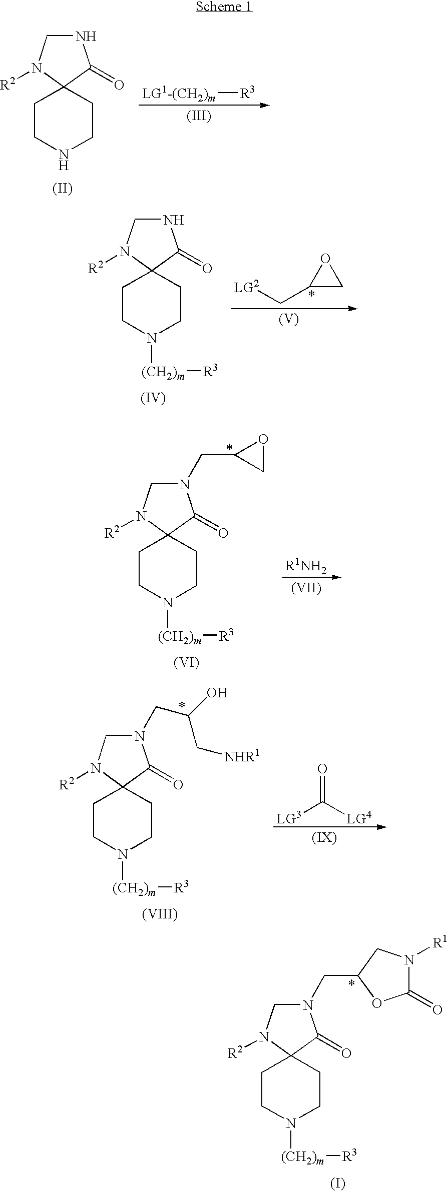

Accordingly, a suitably substituted compound of formula (II), a known compound or compound prepared by known methods, is reacted with a suitably substituted compound of formula (III), wherein LG¹ is a suitably selected leaving group such as Cl, Br, I, triflate, and the like, a known compound or compound prepared by known methods, in the presence of a base such as TEA, DIPEA, pyridine, Na₂CO₃, K₂CO₃, and the like, wherein the base is preferably present in an amount equal to or greater than about one molar equivalent, in an organic solvent such as DMF, DMSO, NMP, and the like, to yield the corresponding compound of formula (IV).

The compound of formula (IV) is reacted with a suitable substituted compound of formula (V) wherein LG² is a suitably selected leaving group such as Cl, Br, I, tosylate, mesylate, and the like, a known compound or compound prepared by known methods, in the presence of an inorganic base such as NaH, KO-t-Bu, K₂CO₃, NaHMDS, LiHMDS, and the like, in an organic solvent such as NMP, DMF, THF, and the like, to yield the corresponding compound of formula (VI).

The compound of formula (VI) is reacted with a suitably substituted compound of formula (VII), a known compound or compound prepared by known methods, in an organic solvent such as ethanol, acetonitrile, methanol, isopropanol, and the like, to yield the corresponding compound of formula (VIII).

The compound of formula (VIII) is reacted with a suitably substituted compound of formula (IX), wherein LG³ and LG⁴ are suitably selected leaving groups such as Cl, imidazole, trichloromethylethoxy and the like, a known compound or compound prepared by known methods, in an organic solvent such as dichloromethane, acetonitrile, and the like, to yield the corresponding compound of formula (I). Preferably, LG³ and LG⁴ are the same, more preferably, LG³ and LG⁴ are each Cl.

One skilled in the art will recognize that in the preparation of compounds of formula (I) and (VIII) as described in Scheme 1 above (i.e. in reactions where the oxarinyl group is opened with a suitably substituted compound of formula (VII)), the stereo-configuration of the hydroxy group in the product will be determined by the stereo-configuration of the compound of formula (V). For example, where the compound of formula (V) is (R)-oxarinyl-methyl, then the compound of formula (I) is prepared in the (R) configuration at the “*” stereocenter.

Compounds of formula (Ib) (compounds of formula (I) wherein R¹ is other than hydrogen) may alternatively be prepared as outlined in Scheme 2, below.

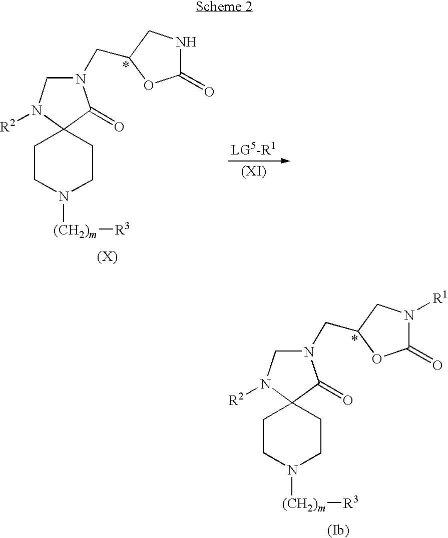

Accordingly, a suitably substituted compound of formula (X) (where the compound of formula (X) (a compound of formula (I) wherein R¹ is H), is reacted with a suitably substituted compound of formula (XI) wherein LG⁵ is a suitably selected leaving group such as Cl, Br, I, triflate, and the like, a known compound or compound prepared by known methods, in the presence of an inorganic base such as NaH, KO-t-Bu, NaHMDS, LiHMDS, and the like, in an organic solvent such as NMP, DMF, THF, and the like, to yield the corresponding compound of formula (Ib) (a compound of formula (I) wherein R¹ is other than hydrogen).

Compounds of formula (I) may alternatively be prepared as outlined in Scheme 3, below.

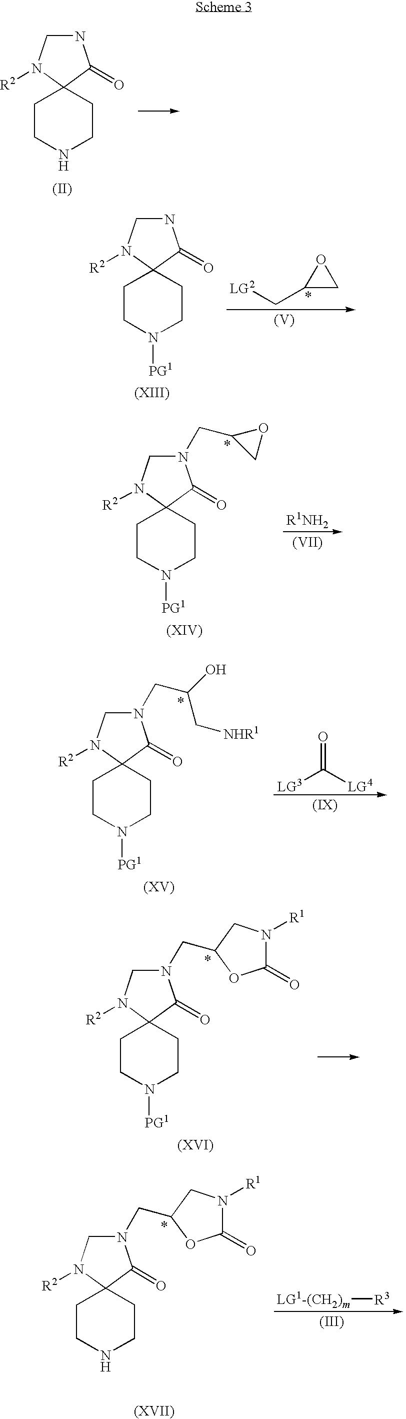

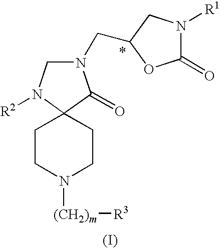

Accordingly, a suitably substituted compound of formula (II), is protected according to known methods, to yield the corresponding compound of formula (XIII), wherein PG¹ is the corresponding nitrogen protecting group, such as t-butoxycarbonyl (BOC), CBz, Fmoc, benzhydryl, triphenylmethyl, 4-methoxybenzyl, benzoyl, and the like. For example, the compound of formula (II) may be reacted with BOC-anhydride, to yield the corresponding compound of formula (XIII), wherein PG¹ is BOC; in another example, the compound of formula (II) may be reacted with benzoyl chloride, to yield the corresponding compound of formula (XIII), wherein PG¹ is benzoyl.

The compound of formula (XIII) is reacted with a suitable substituted compound of formula (V) wherein LG² is a suitably selected leaving group such as Cl, Br, I, tosylate, mesylate, and the like, a known compound or compound prepared by known methods, in the presence of a base such as NaH, KO-t-Bu, K₂CO₃, NaHMDS, LiHMDS, and the like, in an organic solvent such as NMP, DMF, THF, and the like, to yield the corresponding compound of formula (XIV).

The compound of formula (XIV) is reacted with a suitably substituted compound of formula (VI I), a known compound or compound prepared by known methods, in an organic solvent such as ethanol, acetonitrile, methanol, isopropanol, and the like, to yield the corresponding compound of formula (XV).

The compound of formula (XV) is reacted with a suitably substituted compound of formula (IX), wherein LG³ and LG⁴ are suitably selected leaving groups such as Cl, imidazole, trichloromethylethoxy and the like, preferably LG³ and LG⁴ are the same, more preferably LG³ and LG⁴ are the same and are each Cl, a known compound or compound prepared by known methods, in an organic solvent such as dichloromethane, acetonitrile, and the like, to yield the corresponding compound of formula (XVI).

The compound of formula (XVI) is de-protected accordingly to known methods, to yield the corresponding compound of formula (XVII). For example, wherein PG¹ is Boc, the compound of formula (XVI) is de-protected by reacting with an acid such as TFA, at room temperature, in an organic solvent such as dichloromethane, to yield the corresponding compound of formula (XVII).

The compound of formula (XVII) is reacted with a suitably substituted compound of formula (III), wherein LG¹ is a suitably selected leaving group such as Cl, Br, I, triflate, and the like, a known compound or compound prepared by known methods, in the presence of a base such as TEA, DIPEA, pyridine, Na₂CO₃, K₂CO₃, and the like, wherein the base is preferably present in an amount equal to or greater than about one molar equivalent, in an organic solvent such as DMF, DMSO, NMP, and the like, to yield the corresponding compound of formula (I).

One skilled in the art will recognize that in the preparation of compounds of formula (I) and (XV) as described in Scheme 3 above (i.e. in reactions where the oxarinyl group is opened with a suitably substituted compound of formula (VII)), the stereo-configuration of the hydroxy group will be determined by the stereo-configuration of the compound of formula (V). For example, where the compound of formula (V) is (R)-oxarinyl-methyl, then the compound of formula (I) is prepared in the (R) configuration at the “*” stereocenter.

Compounds of formula (I) may alternatively be prepared as outlined in Scheme 4, below

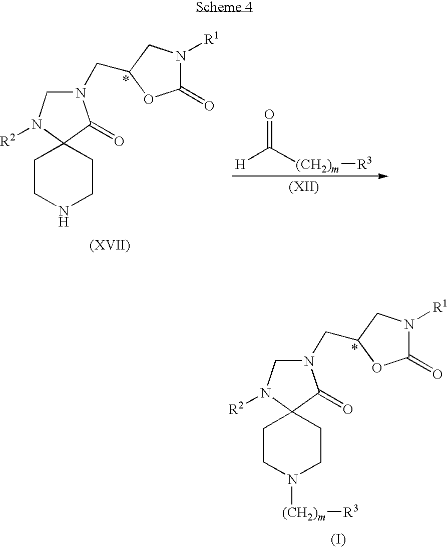

Accordingly, a suitably substituted compound of formula (XVII), a known compound or compound prepared by known methods, is reacted with a suitably substituted compound of formula (XII), a known compound or compound prepared by known methods, in the presence of a suitably selected reducing agent such as sodium triacetoxyborohydride, sodium cyanoborohydride, and the like, in the presence of an acid such as acetic acid, titanium tetra-isopropoxide, boron trifluoride, and the like, in an organic solvent such as DCE, THF, acetonitrile, and the like, to yield the corresponding compound of formula (I).

Compounds of Formula (I) may alternatively be prepared as outlined in Scheme 5, below.

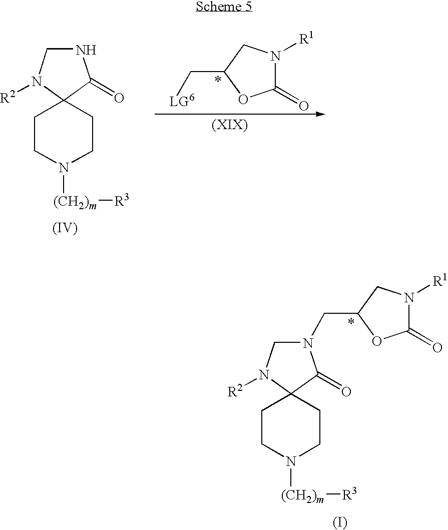

Accordingly, a suitably substituted compound of formula (IV) is reacted with a suitably substituted compound of formula (XIX) wherein LG⁶ is a suitably selected leaving group such as Cl, Br, I, tosylate, mesylate, and the like, a known compound or compound prepared by known methods, in the presence of an inorganic base such as NaH, KO-t-Bu, NaHMDS, LiHMDS, and the like, in an organic solvent such as NMP, DMF, THF, and the like, to yield the corresponding compound of formula (I).

Intermediate compounds of formula (IV) may be prepared as outlined in Scheme 6, below.

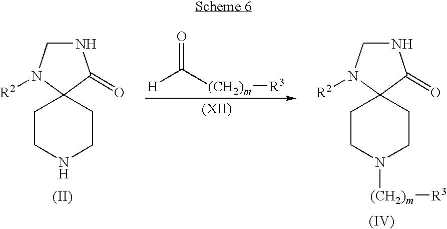

Accordingly, a suitably substituted compound of formula (II), a known compound or compound prepared by known methods, is reacted with a suitably substituted compound of formula (XII), a known compound or compound prepared by known methods, in the presence of a suitably selected reducing agent such as sodium triacetoxyborohydride, sodium cyanoborohydride, and the like, in the presence of an acid such as acetic acid, titanium tetra-isopropoxide, boron trifluoride, and the like, in an organic solvent such as DCE, THF, acetonitrile, and the like, to yield the corresponding compound of formula (IV).

Intermediate compounds of formula (XVI) may be prepared as outlined in Scheme 7, below.

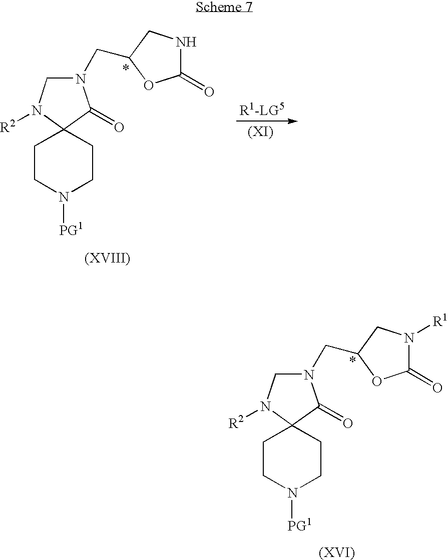

Accordingly, suitably substituted compound of formula (XVIII) (a compound of formula (XVI) wherein R¹ is H, prepared as in, for example, Scheme 4), is reacted with a suitably substituted compound of formula (XI), wherein LG⁵ is a suitably selected leaving group such as Cl, Br, I, triflate, and the like, a known compound or compound prepared by known methods, in the presence of a base such as as NaH, KO-t-Bu, K₂CO₃, NaHMDS, LiHMDS, and the like, in an organic solvent such as NMP, DMF, THF, and the like, to yield the corresponding compound of formula (XVI).

Intermediate compounds of formula (XVI) may alternatively be prepared as outlined in Scheme 8, below.

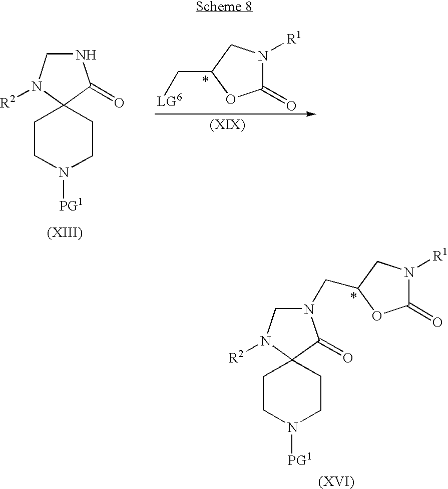

Accordingly, a suitably substituted compound of formula (XII I) is reacted with a suitably substituted compound of formula (XIX) wherein LG⁶ is a suitably selected leaving group such as Cl, Br, I, tosylate, mesylate, and the like, a known compound or compound prepared by known methods, in the presence of an inorganic base such as NaH, KO-t-Bu, NaHMDS, LiHMDS, and the like, in an organic solvent such as NMP, DMF, THF, and the like, to yield the corresponding compound of formula (XVI).

The present invention further comprises pharmaceutical compositions containing one or more compounds of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01-1000 mg or any range therein, and may be given at a dosage of from about 0.01-300 mg/kg/day, or any range therein, preferably from about 0.5-100 mg/kg/day, or any range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 1000 mg, or any range therein, of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating disorders and conditions mediated by the ORL-1 receptor described in the present invention may also be carried out using a pharmaceutical composition comprising any of the compounds as defined herein and a pharmaceutically acceptable carrier. The pharmaceutical composition may contain between about 0.01 mg and 1000 mg of the compound, or any range therein; preferably about 10 to 500 mg of the compound, or any range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

The compound of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phophatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamidephenol, polyhydroxyethylaspartamidephenol, or polyethyl eneoxidepolylysine substituted with palmitoyl residue. Furthermore, the compounds of the present invention may be coupled to a class of biodegradable polymers useful in achieving controlled release of a drug, for example, polylactic acid, polyepsilon caprolactone, polyhydroxy butyeric acid, polyorthoesters, polyacetals, polydihydropyrans, polycyanoacrylates and cross-linked or amphipathic block copolymers of hydrogels.

To prepare a pharmaceutical composition of the present invention, a compound of formula (I) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of the present invention may be administered in any of the foregoing compositions and according to dosage regimens established in the art whenever treatment of ORL-1 disorders is required.

The daily dosage of the products may be varied over a wide range from 0.01 to 10,000 mg per adult human per day, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.01 mg/kg to about 300 mg/kg of body weight per day, or any range therein. Preferably, the range is from about 0.5 to about 100.0 mg/kg of body weight per day, or any range therein. More preferably, from about 1.0 to about 50.0 mg/kg of body weight per day, or any range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### Example 1

### 8-(R)-Acenaphthen-1-yl-1-(4-fluoro-phenyl)-3-(S)-(2-oxo-oxazolidin-5-ylmethyl)-1,3,8-triaza-spiro[4.5]decan-4-one

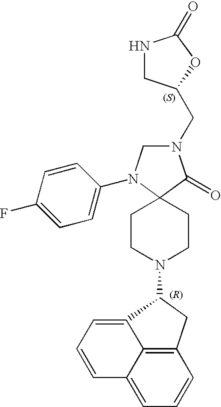

(R)-8-Acenaphthen-1-yl-3-(3-amino-2-hydroxy-(S)-propyl)-1-(4-fluoro-phenyl)-1,3,8-triaza-spiro[4.5]decan-4-one (0.05 g, 0.105 mmol) was dissolved in dry dichloromethane (3.5 mL). To the solution was then added at 0° C. triethylamine (0.032 g, 0.31 mmol). The reaction mixture was stirred at 0° C. for 10 minutes. To the resulting mixture was then added at 0° C. a solution of trisphosgene (0.015 g, 0.052 mmol) in dry dichloromethane (0.75 mL). The resulting mixture was stirred at 0° C. for 1 hour under nitrogen atmosphere. The resulting mixture was then partitioned with aqueous 1N HCl and dichloromethane. The organic layer was washed with brine, dried with Na₂SO₄, filtered and the solvent evaporated in vacuo to yield a solid. The solid was purified via flash chromatography (5.0% ammonia 2.0 M in methanol/dichloromethane) to yield the title compound.

¹H NMR (400 MHz, CDCl₃) δ7.68-7.66 (1H, m), 7.61-7.59 (1H, m), 7.53-7.42 (3H, m), 7.26-7.25 (1H, m), 7.04-6.98 (4H, m), 5.01-4.93 (2H, m), 4.88-4.81 (1H, m), 4.79-4.77 (1H, m), 4.73-4.71 (1H, m), 3.80-3.67 (2H, m), 3.59-3.32 (4H, m), 3.09-2.98 (2H, m), 2.84-2.75 (1H, m), 2.48-2.41 (1H, m), 2.35-2.29 (1H, m), 2.27-2.15 (1H, m), 1.76-1.63 (2H, m)

MS (ES⁺) m/z 501.1 (M+H)⁺.

### Example 2

### 1-(4-Fluoro-phenyl)-8-(8-methyl-naphthalen-1-ylmethyl)-3-(R)-(2-oxo-oxazolidin-5-ylmethyl)-1,3,8-triaza-spiro[4.5]decan-4-one

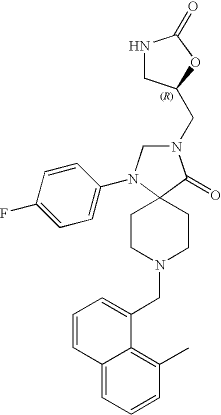

3-(3-Amino-2-(R)-hydroxy-propyl)-1-(4-fluoro-phenyl)-8-(8-methyl-naphthalen-1-ylmethyl)-1,3,8-triaza-spiro[4.5]decan-4-one (0.100 g, 0.209 mmol) was dissolved in dry dichloromethane (7 mL). To the resulting solution was then added at 0° C. triethylamine (0.063 g, 0.63 mmol). The resulting mixture was stirred at 0° C. for 10. To the resulting mixture was then added at 0° C. a solution of trisphosgene (0.031 g, 0.105 mmol) in dry dichloromethane (1 mL). The resulting mixture was stirred at 0° C. for 1 hour under nitrogen atmosphere. The resulting mixture was then partitioned with aqueous 1N HCl and dichloromethane. The organic layer was washed with brine, dried with Na₂SO₄, filtered and the solvent evaporated in vacuo to yield a solid. The solid was purified via flash chromatography (6.0% ammonia 2.0 M in methanol/dichloromethane) to yield the title compound as a white solid.

¹H NMR (300 MHz, CDCl₃) δ7.77 (1H, dd, J=2.05 and 7.5 Hz), 7.71-7.68 (1H, m), 7.37-7.29 (4H, m), 6.98-6.85 (4H, m), 5.07 (1H, bs), 4.92-4.84 (1H, m), 4.75 (2H, dd, J=5.0 and 17.1 Hz), 3.99 (2H, s), 3.84-3.71 (2H, m), 3.62-3.55 (1H, m), 3.48-3.42 (1H, m), 3.09 (3H, s), 2.82-2.75 (4H, m), 2.25-2.15 (2H, m), 1.69-1.64 (2H, m)

MS (ES⁺) m/z 503.1 (M+H)⁺.

### Example 3

### 8-Cyclooctylmethyl-1-(4-fluoro-phenyl)-3-(R)-(2-oxo-oxazolidin-5-ylmethyl)-1,3,8-triaza-spiro[4.5]decan-4-one

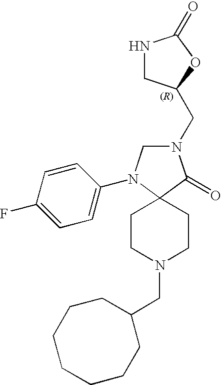

3-(3-Amino-2-hydroxy-propyl)-8-cyclooctylmethyl-1-(4-fluoro-phenyl)-1,3,8-triaza-spiro[4.5]decan-4-one (0.100 g, 0.22 mmol) was dissolved in dry dichloromethane (20 mL). To the resulting solution was then added at 0° C. trisphosgene (0.066 g, 0.22 mmol). The resulting mixture was stirred at 0° C. for 1 hour and at room temperature for 1 hour under nitrogen atmosphere. The resulting mixture was then partitioned with aqueous 1N NaOH and dichloromethane. The organic layer was washed with brine, dried with Na₂SO₄, filtered and the solvent evaporated in vacuo to yield an oil. The oil was purified via flash chromatography (4.0% ammonia 2.0 M in methanol/dichloromethane) to yield the title compound as a gum, which was then triturated with n-hexanes to yield a residue.

¹H NMR (300 MHz, CDCl₃) δ 7.03-6.91 (4H, m), 4.94-4.83 (1H, m), 4.79-4.72 (2H, m), 3.83-3.70 (2H, m), 3.63-3.55 (1H, m), 3.47-3.41 (1H, m), 2.66-2.62 (4H, m), 2.28-2.17 (2H, m), 2.15-2.09 (2H, m), 1.70-1.39 (18H, m)

MS (ES⁺) m/z 473.4 (M+H)⁺.

### Example 4

### 1-(4-Fluoro-phenyl)-8-(8-methyl-naphthalen-1-ylmethyl)-3-(S)-(3-methyl-2-oxo-oxazolidin-5-ylmethyl)-1,3,8-triaza-spiro[4.5]decan-4-one

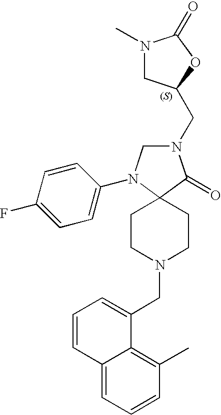

1-(4-Fluoro-phenyl)-8-(8-methyl-naphthalen-1-ylmethyl)-3-(R)-(2-oxo-oxazolidin-5-ylmethyl)-1,3,8-triaza-spiro[4.5]decan-4-one (0.03 g, 0.06 mmol) was dissolved in N,N-dimethylformamide (1.5 mL). To the resulting mixture was then added at room temperature sodium hydride (60% in mineral oil, 3 mg, 0.07 mmol) under nitrogen atmosphere and the resulting mixture was stirred at room temperature for 5 minutes, then at 40° C. for one hour, then cooled down to room temperature. To the resulting mixture was then added at room temperature methyl iodide (0.011 g, 0.07 mmol). The resulting mixture was stirred at 40° C. for 6 hours, then at room temperature under nitrogen atmosphere for 18 hours and then partitioned with water and dichloromethane. The organic layer was dried with Na₂SO₄, filtered and the solvent evaporated in vacuo to yield an oil. The oil was purified via flash chromatography (4% ammonia 2.0 M in methanol/dichloromethane) to yield the title compound.

¹H NMR (300 MHz, CDCl₃) δ7.79-7.76 (1H, m), 7.72-7.68 (1H, m), 7.37-7.32 (4H, m), 6.98-6.85 (4H, m), 4.77-4.70 (3H, m), 4.00 (2H, bs), 3.82-3.76 (1H, m), 3.70-3.64 (1H, m), 3.59-3.52 (1H, m), 3.48-3.37 (1H, m), 3.08 (3H, s), 2.87 (3H, s), 2.83-2.69 (4H, m), 2.28-2.12 (2H, m), 1.70-1.60 (4H, m)

MS (ES⁺) m/z 517.2 (M+H)⁺.

### Example 5

### 1-(4-Fluoro-phenyl)-3-(R)-[3-(2-methoxy-ethyl)-2-oxo-oxazolidin-5-ylmethyl]-8-(8-methyl-naphthalen-1-ylmethyl)-1,3,8-triaza-spiro[4.5]decan-4-one

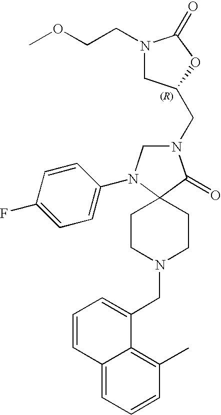

1-(4-Fluoro-phenyl)-8-(8-methyl-naphthalen-1-ylmethyl)-3-(R)-(2-oxo-oxazolidin-5-ylmethyl)-1,3,8-triaza-spiro[4.5]decan-4-one (0.043 g, 0.086 mmol) was dissolved in N,N-dimethylformamide (1.5 mL). To the resulting mixture was then added at room temperature sodium hydride (60% in mineral oil, 6 mg, 0.138 mmol) under nitrogen atmosphere and the resulting mixture was stirred at room temperature for 5 minutes, then at 40° C. for 45 minutes, then cooled down to room temperature. To the resulting mixture was then added at room temperature 1-bromo-2-methoxy-ethane (0.024 g, 0.17 mmol). The resulting mixture was stirred at 60° C. for 48 hours under nitrogen atmosphere for 18 hours and partitioned with water and ethyl acetate. The organic layer was dried with Na₂SO₄, filtered and the solvent evaporated in vacuo to yield an oil. The oil was purified via flash chromatography (4% ammonia 2.0 M in methanol/dichloromethane) to yield the title compound.

¹H NMR (300 MHz, CDCl₃) δ7.78-7.76 (1H, m), 7.71-7.69 (1H, m), 7.37-7.31 (4H, m), 6.97-6.85 (4H, m), 4.79-4.69 (3H, m), 4.00 (2H, bs), 3.82-3.77 (2H, m), 3.70-3.64 (1H, m), 3.56-3.34 (7H, m), 3.1 (3H, s), 2.81-2.76 (4H, m), 2.25-2.15 (2H, m), 1.68-1.63 (4H, m)

MS (ES⁺) m/z 561.2 (M+H)⁺.

### Example 6

### Filtration Binding Assays: ORL-1, Mu, Kappa and Delta Opioid Receptors

The assays used to measure the binding of representative test compounds to the ORL-1, delta, kappa and mu opioid receptors were run similarly, with appropriate selection and substitution of cell membrane and radiolabeled ligand. The following cell membranes and ligands were used for the determination of binding to the respective opioid receptors.
ORL-1 (Nociceptin) 1 ug/well of 3C4 cell line membrane and 0.5 nM final concentration of ¹²⁵I nociceptin
Delta (δ) opioid: 1 μg/well of 2D4 cell line membrane and a final concentration of 2.44 nM DPDPE-³H ligand.
Mu (μ) opioid: 5 μg/well of 1D4 cell line membrane and a final concentration 0.8993 nM DAMGO-³H ligand.
Kappa (κ) opioid: 7 μg/well of 2C2 cell line membrane and a final concentration of 2.76 nM U-69,593-³H ligand.

Both membrane and ligand were diluted such that a 25 μl addition delivered the necessary amount per well, as noted above. Both membrane and ligand were diluted in 1× ORL-1 buffer. The ORL-1 buffer was composed of 50 mM Tris-HCl, pH=7.4, 5 mM MgCl₂ and 1 mM EGTA. Each test compound was diluted to a concentration in the range of from 100 μM to 10 pM (half-log curve) with 100% DMSO. To each well of a 96 well plate was added 25 μL cell membrane (as listed above), 1 μL of the diluted test compound, and 25 μL labeled ligand (as listed above) for the mu, delta, kappa or ORL-1 opioid receptor, as desired.

The plate was incubated on a rotating shaker for 2 hours at room temperature. The plate was filtered over GF/C Filterplates, prewetted in 0.03% polyethyleneimine, in Filtermate 196 apparatus (Packard). The plate was then washed 6 times with ORL-1 buffer in the filtration apparatus and dried in vacuum oven for 1 hour at a temperature of 50° C.

To each well was then added 25 μL Microscint 20 (Packard) (to solubilize bound radioactivity) and each well counted in a Packard TopCount for 1 minute/well using counting parameters optimized for the particular radioligand/opioid receptor being tested. Percent radioactive ligand bound in each reaction was calculated relative to a control using DMSO for maximum binding (no inhibition). Curves were fitted and Kᵢ's determined using Graphpad Prizm software (v3.0). The Kᵢs were calculated using the following formula by Graphpad Prizm software, where

Ki=(IC₅₀)/(1+[radioligand]/K_{d})

For the ORL-1, the K_{d} is 0.5 nM, for Mu the K_{d} is 0.8993 nM, for kappa the K_{d} is 2.76 nM and for delta the K_{d} is 2.44 nM. Note that the [radioligand] (concentration of radioligand) was equivalent to the K_{d}.

Representative compounds of the present invention were tested for binding to the ORL-1, mu, kappa and delta opioid receptors using the procedure, cell membranes and ligands as described above, with results as listed in Table 2. The values listed below correspond to IC₅₀ or Ki measurement as denoted at the top of the column, unless otherwise noted. For the compounds which were tested more than once, the value listed in Table 2 is the calculated mean.

TABLE 2

| | ORL-1 | ORL-1 | Delta | Kappa | Mu |
|---|---|---|---|---|---|
| ID# | IC₅₀ (nM) | Ki (nM) | Ki (μM) | Ki (μM) | Ki (μM) |
| 1 | 0.55 | NT | NT | IC₅₀ = 0.51 | IC₅₀ = 0.16 |
| 2 | NT | 12 | >5 | 0.64 | 0.74 |
| 3 | NT | 58 | >5 | 0.13 | 0.06 |
| 4 | NT | 23 | >5 | 0.12 | 0.36 |
| 5 | NT | 49 | >5 | 0.58 | 0.18 |

NT = not tested

### Example 7

### Oral Formulation (Prophetic Example)

As a specific embodiment of an oral composition, 100 mg of the compound prepared as in Example 2 is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A compound of formula (I) 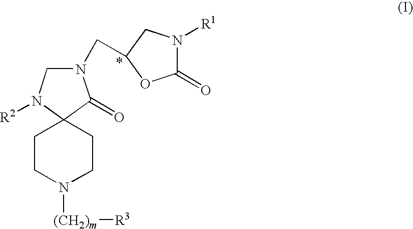
wherein
R¹ is selected from the group consisting of hydrogen, C₁₋₄alkyl, —C₁₋₄alkyl-OH and —C₁₋₄alkyl-O—C₁₋₄alkyl;
R² is phenyl; wherein the phenyl is optionally substituted with one or more substituents independently selected from the group consisting of halogen, hydroxyl, C₁₋₄alkyl and C₁₋₄alkoxy;
m is an integer from 0 to 1;
R³ is selected from the group consisting of cycloalkyl, partially unsaturated carbocyclyl, aryl and heteroaryl; wherein the aryl or heteroaryl is optionally substituted with one or more substituents independently selected from the group consisting of halogen, hydroxyl, C₁₋₄alkyl and C₁₋₄alkoxy;
or an enantiomer, diastereoisomer, ester, prodrug, solvate or pharmaceutically acceptable salt thereof.

2. A compound as in claim 1, wherein
R¹ is selected from the group consisting of hydrogen, C₁₋₂alkyl, C₁₋₂alkyl-OH and —CH₂CH₂OCH₃;
R² is phenyl; wherein the phenyl is optionally substituted with one to three halogen;
m is an integer from 0 to 1;
R³is selected from the group consisting of cycloalkyl, partially unsaturated carbocyclyl and aryl; wherein the aryl is optionally substituted with C₁₋₂alkyl;
or an enantiomer, diastereoisomer, ester, prodrug, solvate or pharmaceutically acceptable salt thereof.

3. A compound as in claim 2, wherein
R¹ is selected from the group consisting of hydrogen, C₁₋₂alkyl and —CH₂CH₂OCH₃;
R²is 4-fluorophenyl;
m is an integer from 0 to 1;
R³is selected from the group consisting of cycloalkyl, partially unsaturated carbocyclyl and aryl; wherein the aryl is optionally substituted with C₁₋₂alkyl;
or an enantiomer, diastereoisomer, ester, prodrug, solvate or pharmaceutically acceptable salt thereof.

4. A compound as in claim 3, wherein
R¹ is selected from the group consisting of hydrogen, methyl and —CH₂CH₂OCH₃;
R²is 4-fluorophenyl;
m is an integer from 0 to 1;
R³is selected from the group consisting of cyclooctyl, acenaphthenyl and 8-methyl-napth-1-yl;
or an enantiomer, diastereoisomer, ester, prodrug, solvate or pharmaceutically acceptable salt thereof.

5. A compound as in claim 4, wherein
R¹ is selected from the group consisting of hydrogen, methyl and —CH₂CH₂OCH₃;
R²is 4-fluorophenyl;
m is 1;
R³ is selected from the group consisting of cyclooctyl and 8-methyl-naphth-1-yl;
or an enantiomer, diastereoisomer, ester, prodrug, solvate or pharmaceutically acceptable salt thereof

6. A compound as in claim 4, selected from the group consisting of
8-(R)-Acenaphthen-1-yl-1-(4-fluoro-phenyl)-3-(S)-(2-oxo-oxazolidin-5-ylmethyl)-1,3,8-triaza-spiro[4.5]decan-4-one;
1-(4-Fluoro-phenyl)-8-(8-methyl-naphthalen-1-ylmethyl)-3-(R)-(2-oxo-oxazolidin-5-ylmethyl)-1,3,8-triaza-spiro[4.5]decan-4-one;
8-Cyclooctylmethyl-1-(4-fluoro-phenyl)-3-(2-oxo-oxazolidin-5-ylmethyl)-1,3,8-triaza-spiro[4.5]decan-4-one;
1-(4-Fluoro-phenyl)-8-(8-methyl-naphthalen-1-ylmethyl)-3-(S)-(3-methyl-2-oxo-oxazolidin-5-ylmethyl)-1,3,8-triaza-spiro[4.5]decan-4-one; and
1-(4-Fluoro-phenyl)-3-[3-(2-methoxy-ethyl)-2-oxo-oxazolidin-5-ylmethyl]-8-(8-methyl-naphthalen-1-ylmethyl)-1,3,8-triaza-spiro[4.5]decan-4-one,
and enantiomers, diastereoisomers, esters, prodrugs, solvates and pharmaceutically acceptable salts thereof.

7. A compound as in claim 1, selected from the group consisting of
8-(R)-Acenaphthen-1-yl-1-(4-fluoro-phenyl)-3-(S)-(2-oxo-oxazolidin-5-ylmethyl)-1,3,8-triaza-spiro[4.5]decan-4-one,
and enantiomer, diastereoisomer, ester, prodrug, solvate or pharmaceutically acceptable salt thereof

8. A pharmaceutical composition comprising a pharmaceutically acceptable carrier and a compound as in claim 1.

9. A pharmaceutical composition made by mixing at least one compound as in claim 1 and at least one pharmaceutically acceptable carrier.

10. A process for making a pharmaceutical composition comprising mixing a compound as in claim 1 and a pharmaceutically acceptable carrier.

11. A method for treating, preventing or ameliorating a disorder or condition mediated by the ORL-1 receptor, comprising administering to a subject in need thereof a therapeutically effective amount of a compound as in claim 1.

12. The method of claim 11, wherein the disorder mediated by the ORL-1 receptor is selected from the group consisting of anxiety, depression, panic, mania, dementia, bipolar disorder, substance abuse, neuropathic pain, acute pain, chronic pain, migraine, asthma, cough, psychosis, schizophrenia, epilepsy, hypertension, obesity, eating disorders, cravings, diabetes, cardiac arrhythmia, irritable bowel syndrome, Crohn's disease, urinary incontinence, adrenal disorders, attention deficit disorder (ADD), attention deficit hyperactivity disorder (ADHD), Alzheimer's disease, for improved cognition or memory and mood stabilization.

13. A method of treating, preventing or ameliorating a disorders or condition mediated by the ORL-1 receptor, comprising administering to a subject in need thereof a therapeutically effective amount of a composition as in claim 8.

14. A method of treating a condition selected from the group consisting of anxiety, depression, panic, mania, dementia, bipolar disorder, substance abuse, neuropathic pain, acute pain, chronic pain, migraine, asthma, cough, psychosis, schizophrenia, epilepsy, hypertension, obesity, eating disorders, cravings, diabetes, cardiac arrhythmia, irritable bowel syndrome, Crohn's disease, urinary incontinence, adrenal disorders, attention deficit disorder (ADD), attention deficit hyperactivity disorder (ADHD), Alzheimer's disease, for improved cognition or memory and mood stabilization comprising administering to a subject in need thereof a therapeutically effective amount of a compound as in claim 1.

15. A method of treating a condition selected from the group consisting of anxiety, depression, panic, mania, dementia, bipolar disorder, substance abuse, neuropathic pain, acute pain, chronic pain, migraine, asthma, cough, psychosis, schizophrenia, epilepsy, hypertension, obesity, eating disorders, cravings, diabetes, cardiac arrhythmia, irritable bowel syndrome, Crohn's disease, urinary incontinence, adrenal disorders, attention deficit disorder (ADD), attention deficit hyperactivity disorder (ADHD), Alzheimer's disease, for improved cognition or memory and mood stabilization comprising administering to a subject in need thereof a therapeutically effective amount of a composition as in claim 8.

16. The use of a compound as in claim 1 for the preparation of a medicament for treating: (a) anxiety, (b) depression, (c) panic, (d) mania, (e) dementia, (f) bipolar disorder, (g) substance abuse, (h) neuropathic pain, (i) acute pain, (j) chronic pain, (k) migraine, (l) asthma, (m) cough, (n) psychosis, (o) schizophrenia, (p) epilepsy, (q) hypertension, (r) obesity, (s) eating disorders, (t) cravings, (u) diabetes, (v) cardiac arrhythmia, (w) irritable bowel syndrome, (x) Crohn's disease, (y) urinary incontinence, (z) adrenal disorders, (aa) attention deficit disorder (ADD), (bb) attention deficit hyperactivity disorder (ADHD), (cc) Alzheimer's disease, for (dd) improved cognition, (ee) improved memory or (ff) mood stabilization, in a subject in need thereof.

17. A process for the preparation of a compound of formula (I) 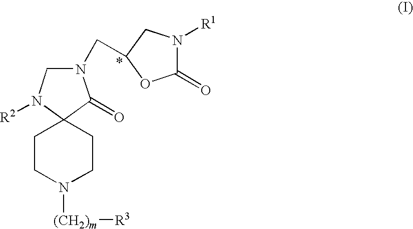
wherein
R¹ is selected from the group consisting of hydrogen, C₁₋₄alkyl, —C₁₋₄alkyl-OH and —C₁₋₄alkyl-O—C₁₋₄alkyl;
R² is phenyl; wherein the phenyl is optionally substituted with one or more substituents independently selected from the group consisting of halogen, hydroxyl, C₁₋₄alkyl and C₁₋₄alkoxy;
m is an integer from 0 to 1;
R³ is selected from the group consisting of cycloalkyl, partially unsaturated carbocyclyl, aryl and heteroaryl; wherein the aryl or heteroaryl is optionally substituted with one or more substituents independently selected from the group consisting of halogen, hydroxyl, C₁₋₄alkyl and C₁₋₄alkoxy;
or a pharmaceutically acceptable salt thereof; comprising 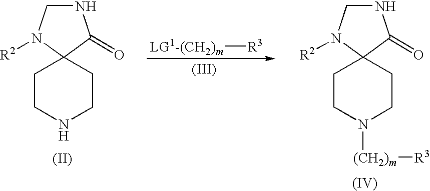
reacting a compound of formula (II) with a compound of formula (III), wherein LG¹ is a leaving group, in the presence of a base, in an organic solvent, to yield the corresponding compound of formula (IV); 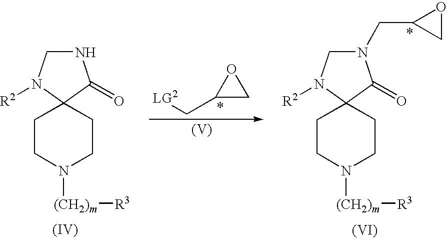
reacting the compound of formula (IV) with a compound of formula (V), wherein LG² is a leaving group, in the presence of an inorganic base, in an organic solvent, to yield the corresponding compound of formula (VI); 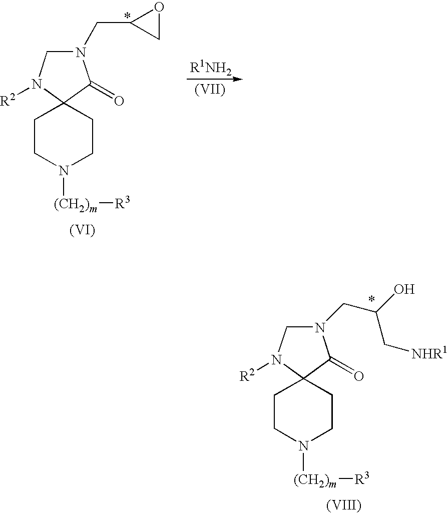
reacting the compound of formula (VI) with a compound of formula (VII), in an organic solvent, to yield the corresponding compound of formula (VIII); 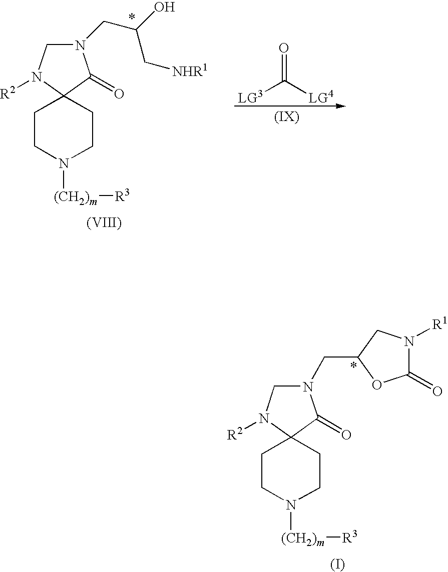
reacting the compound of formula (VIII) with a compound of formula (IX), wherein LG³ and LG⁴ are independently selected leaving groups, in an organic solvent, to yield the corresponding compound of formula (I).

18. A process for the preparation of a compound of formula (Ib) 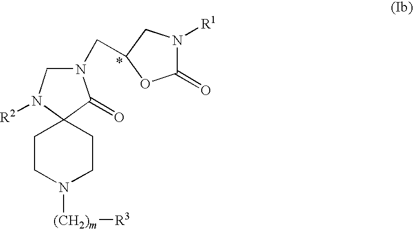
wherein
R¹ is selected from the group consisting of C₁₋₄alkyl, —C₁₋₄alkyl-OH and —C₁₋₄alkyl-O—C₁₋₄alkyl;
R² is phenyl; wherein the phenyl is optionally substituted with one or more substituents independently selected from the group consisting of halogen, hydroxyl, C₁₋₄alkyl and C₁₋₄alkoxy;
m is an integer from 0 to 1;
R³ is selected from the group consisting of cycloalkyl, partially unsaturated carbocyclyl, aryl and heteroaryl; wherein the aryl or heteroaryl is optionally substituted with one or more substituents independently selected from the group consisting of halogen, hydroxyl, C₁₋₄alkyl and C₁₋₄alkoxy;
or a pharmaceutically acceptable salt thereof; comprising 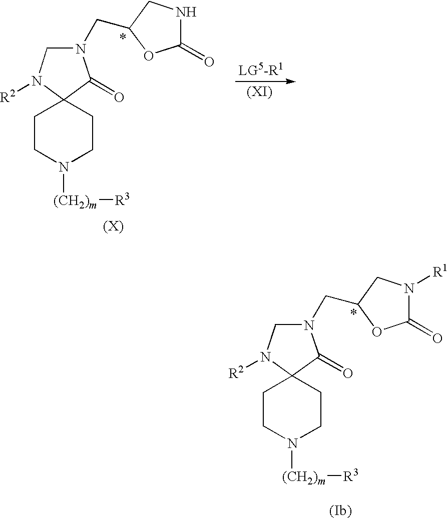
reacting a compound of formula (X) with a compound of formula (XI) wherein LG⁵ is a leaving group, in the presence of an inorganic base, in an organic solvent, to yield the corresponding compound of formula (Ib).

19. A process for the preparation of a compound of formula (I) 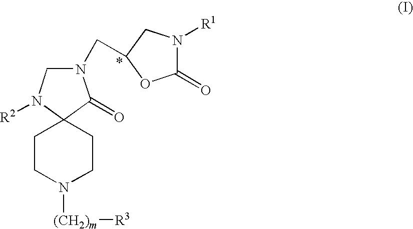
wherein
R¹ is selected from the group consisting of hydrogen, C₁₋₄alkyl, —C₁₋₄alkyl-OH and —C₁₋₄alkyl-O—C₁₋₄alkyl;
R² is phenyl; wherein the phenyl is optionally substituted with one or more substituents independently selected from the group consisting of halogen, hydroxyl, C₁₋₄alkyl and C₁₋₄alkoxy;
m is an integer from 0 to 1;
R³ is selected from the group consisting of cycloalkyl, partially unsaturated carbocyclyl, aryl and heteroaryl; wherein the aryl or heteroaryl is optionally substituted with one or more substituents independently selected from the group consisting of halogen, hydroxyl, C₁₋₄alkyl and C₁₋₄alkoxy;
or a pharmaceutically acceptable salt thereof; comprising 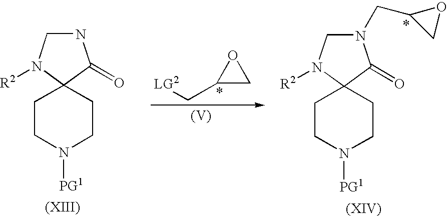
reacting a compound of formula (XIII) with a compound of formula (V), wherein LG² is a leaving group, in the presence of a base, in an organic solvent, to yield the corresponding compound of formula (XIV); 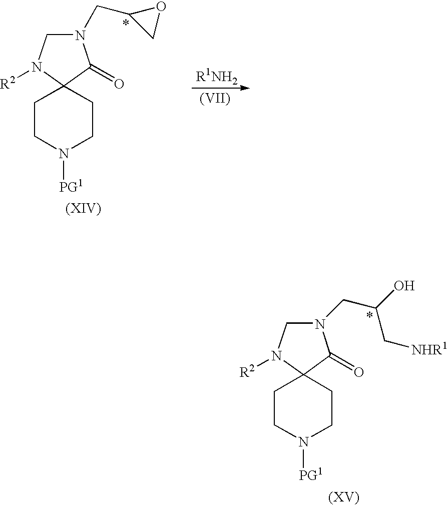
reacting the compound of formula (XIV) with a compound of formula (VII), in an organic solvent, to yield the corresponding compound of formula (XV); 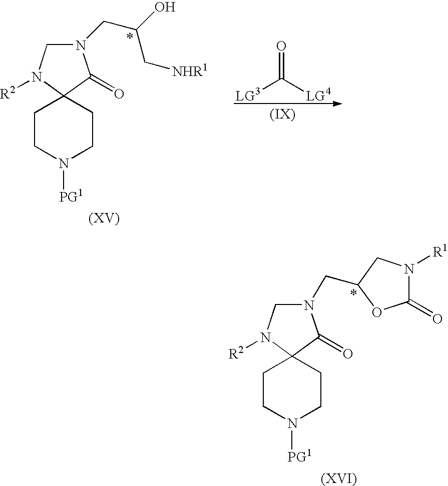
reacting the compound of formula (XV) with a compound of formula (IX), wherein LG³ and LG⁴ are independently selected leaving groups, in an organic solvent, to yield the corresponding compound of formula (XVI); 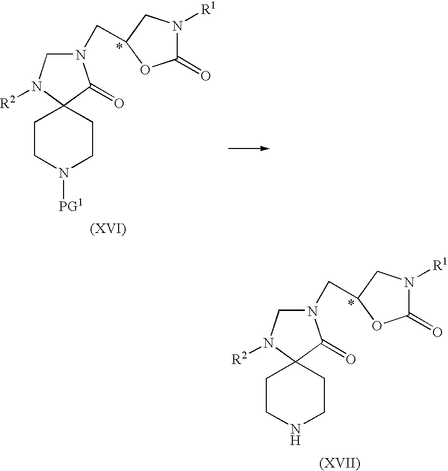
de-protecting the compound of formula (XVI), to yield the corresponding compound of formula (XVII); 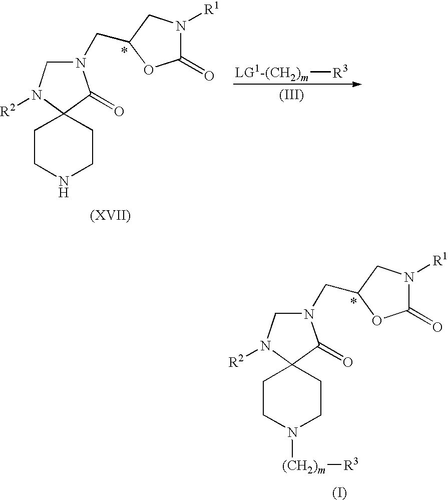
reacting the compound of formula (XVII) with a compound of formula (III), wherein LG¹ is a leaving group, in the presence of a base, in an organic solvent, to yield the corresponding compound of formula (I).

20. A process for the preparation of a compound of formula (I) 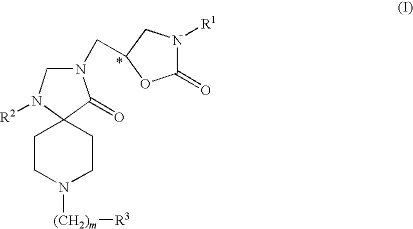
wherein
R¹ is selected from the group consisting of hydrogen, C₁₋₄alkyl, —C₁₋₄alkyl-OH and —C₁₋₄alkyl-O—C₁₋₄alkyl;
R² is phenyl; wherein the phenyl is optionally substituted with one or more substituents independently selected from the group consisting of halogen, hydroxyl, C₁₋₄alkyl and C₁₋₄alkoxy;
m is an integer from 0 to 1;
R³ is selected from the group consisting of cycloalkyl, partially unsaturated carbocyclyl, aryl and heteroaryl; wherein the aryl or heteroaryl is optionally substituted with one or more substituents independently selected from the group consisting of halogen, hydroxyl, C₁₋₄alkyl and C₁₋₄alkoxy;
or a pharmaceutically acceptable salt thereof; comprising 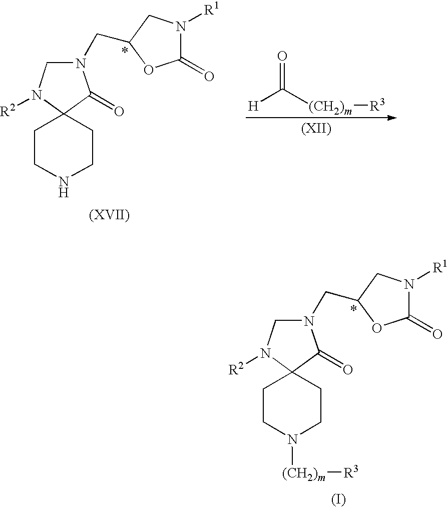
reacting a compound of formula (XVII) with a compound of formula (XII), in the presence of a reducing agent, in the presence of an acid, in an organic solvent, to yield the corresponding compound of formula (I).

21. A process for the preparation of a compound of formula (I) 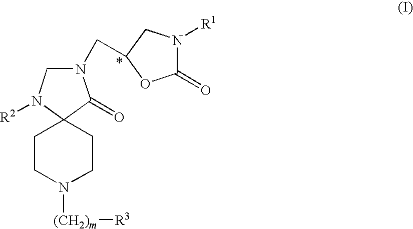
wherein
R¹ is selected from the group consisting of hydrogen, C₁₋₄alkyl, —C₁₋₄alkyl-OH and —C₁₋₄alkyl-O—C₁₋₄alkyl;
R² is phenyl; wherein the phenyl is optionally substituted with one or more substituents independently selected from the group consisting of halogen, hydroxyl, C₁₋₄alkyl and C₁₋₄alkoxy;
m is an integer from 0 to 1;
R³ is selected from the group consisting of cycloalkyl, partially unsaturated carbocyclyl, aryl and heteroaryl; wherein the aryl or heteroaryl is optionally substituted with one or more substituents independently selected from the group consisting of halogen, hydroxyl, C₁₋₄alkyl and C₁₋₄alkoxy;
or a pharmaceutically acceptable salt thereof; comprising 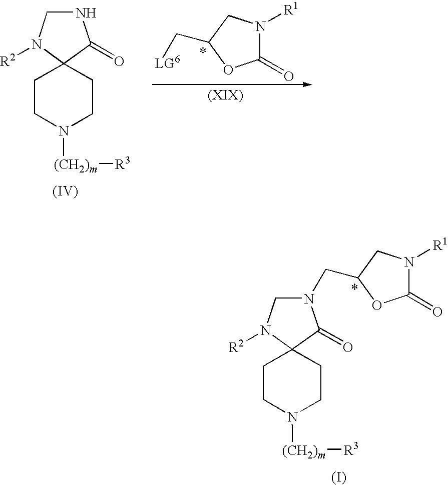
reacting a compound of formula (IV) with a compound of formula (XIX), wherein LG⁶ is a leaving group, in the presence of a base, in an organic solvent, to yield the corresponding compound of formula (I).
